(19) United States
(12) Patent Application Publication
(10) Pub. No.: US 2013/0137586 A1
(43) Pub. Date: May 30, 2013

(54) STABILIZATION OF NUCLEIC ACIDS IN CELL MATERIAL-CONTAINING BIOLOGICAL SAMPLES

(71) Applicants: Christoph Erbacher, Hilden (DE); Markus Kirchmann, Hilden (DE); Patrick Baumhof, Dusslingen (DE); Petrina Schick, Muenchen (DE)

(72) Inventors: Christoph Erbacher, Hilden (DE); Markus Kirchmann, Hilden (DE); Patrick Baumhof, Dusslingen (DE); Petrina Schick, Muenchen (DE)

(73) Assignee: QIAGEN GmbH, Hilden (DE)

(21) Appl. No.: 13/701,439

(22) PCT Filed: Jun. 1, 2011
(86) PCT No.: PCT/EP2011/059162

§ 371 Date: Jan. 30, 2013

(30) Foreign Application Priority Data
Jun. 2, 2010 (EP) 10005711.6

(51) Int. Cl. C12Q 1/68

(52) U.S. Cl. CPC C12Q 1/6806
USPC 506/2; 435/6.12

(57) ABSTRACT

The present invention relates to the use of an aqueous system for stabilizing cell material-containing biological samples while preserving the cell morphology of the cell material and to a method for stabilizing nucleic acids in cell material-containing biological samples while preserving the cell morphology of the cell material.

The present invention relates to the use of a buffer for stabilizing cell material-containing biological samples while preserving the cell morphology of the cell material and to a method for stabilizing nucleic acids in cell material-containing biological samples while preserving the cell morphology of the cell material.

The stabilization of nucleic acids in biological samples is increasingly important in biological, medical and pharmacological research and diagnostics, since the study of the nucleic acids of a cell makes it possible to determine the genetic origin and functional activity thereof. The study of the ribonucleic acids (RNA), more particularly the so-called messenger RNA (mRNA), of a cell allows direct determination of the gene activity of said cell by means of gene expression analysis, which can provide a direct insight into the activity of the cell at the time of collection, since mRNAs, especially of the genes which are transcribed at this time, are present in the cell. A quantitative analysis of the mRNA of a cell by means of modern molecular biology methods such as quantitative or real-time reverse transcriptase polymerase chain reaction (qRT-PCR or real-time RT-PCR) or gene expression chip analyses allows, for example, the identification of expressed genes in order to identify infections, metabolic disorders or cancer. The analysis of the deoxyribonucleic acids (DNA) of a cell by means of molecular biology methods such as PCR or sequencing allows the determination of genetic markers and the detection of genetic defects. Furthermore, the analysis of genomic RNA and DNA can also be used for the direct detection of infectious pathogens such as viruses, bacteria, etc.

An essential requirement for such nucleic acid analysis techniques is the immediate stabilization of nucleic acids immediately after the collection of a biological sample from its natural environment. This applies in particular to RNA, which can be degraded by the ubiquitous and very stable ribonucleases (RNases) immediately after the collection of the biological sample from its environment. Since RNases are very active enzymes which, unlike DNases, do not require any cofactors, even very small amounts of these enzymes suffice for degradation of the majority of the RNA contained in a sample within a very short time.

Moreover, the expression pattern of a cell is subject to a rapid turnover, so that the cell can respond to a change in external conditions. The expression pattern can alter rapidly directly after the sample has been obtained. A drop in temperature, the change in the gas balance and the dilution of the sample by anticoagulants intended to prevent the coagulation of the sample lead to alteration of the expression pattern of the individual cells immediately after collection, especially in the case of blood samples, for example through the induction of stress genes. Only when the ex vivo gene induction has been prevented is it possible to preserve and analyze the in vivo transcription profile in a sample collected from its natural environment. Therefore, especially in the case of medical samples which are collected repeatedly at one location, for example in a doctor's practice, and analyzed in a laboratory only after relatively long storage and transport, stabilization of the nucleic acids is of immense importance.

Various approaches for stabilizing nucleic acids are known from the prior art. The stabilization of RNA in tissue is, for example, achieved by means of ammonium sulfate, which can diffuse rapidly into the cells and reduces transcription and the degradation of RNA by RNases in the cells owing to the denaturation of cellular proteins (so-called RNAlater technology). However, this principle cannot be applied to whole blood, which is one of the most important samples, since whole blood samples contain a high protein content which forms an insoluble precipitate with the reagent. A further widespread method for stabilizing RNA from tissue samples is the use of guanidinium thiocyanate and β-mercaptoethanol, which lyses the cells and denatures the proteins contained therein (D. Gillespie et al. Nucleic Acids Research, 1992, 20 (20), 5492).

Methods for stabilizing RNA in blood, which, in addition to a high content of DNA and proteins, contains in particular various intracellular and extracellular RNases, currently involve the lysis of the cells and a subsequent denaturation of the RNases (U.S. Pat. No. 6,602,718 B1, U.S. Pat. No. 6,617,170 B1 and U.S. Pat. No. 6,821,789). However, a significant disadvantage of these methods, specifically in the case of blood, is that the lysis of the cells leads to mixing of the RNA of different cell types and a large background of undesired RNA thus subsequently complicates the study of the desired RNA. In the case of blood, this is specifically the high amount of mRNA which codes for hemoglobin and originates from the erythrocytes, which are present about 1000 times more often than the leukocytes.

It is therefore an object of the present invention to provide a buffer for stabilizing nucleic acids in cell material-containing biological samples while preserving the cell morphology in order to allow subsequent cell separation, to stabilize the nucleic acids contained in the sample in order to preserve, if possible, the “actual state” in molecular terms at the time of the sample collection, and also to drastically reduce the content of undesired nucleic acids, more particularly undesired RNA, in the sample to be studied after the subsequent lysis of the cells.

It was found that, surprisingly, an aqueous system comprising one or more substances selected from the group consisting of 3-(N-morpholino)propanesulfonic acid (MOPS), 1,2-dimethoxyethane, sodium salicylate, hexaammonium heptamolybdate, glucosamine hydrochloride, indole, 2-(4-hydroxyphenyl)ethanol and tetrahexylammonium chloride stabilizes nucleic acids in cell material-containing biological samples while preserving the cell morphology of the cell material. According to the invention, the “aqueous system” used for this purpose can be any water-based solution, or any buffer, which is suitable for suspending cell material-containing samples or for dissolving parts thereof without denaturing constituents of the sample, provided the solution/buffer contains at least one of the aforementioned substances. The aqueous system according to the invention is capable of stabilizing both intracellular and extracellular nucleic acids in the presence of cell material. In addition, the solutions/buffers according to the invention are also suitable for stabilizing nucleic acids in the presence of free (extracellular) nucleases (DNases and RNases).

Preferably, the aqueous system comprises MOPS or a mixture of MOPS and at least one further substance selected from the group containing 1,2-dimethoxyethane, sodium salicylate, hexaammonium heptamolybdate, glucosamine hydrochloride, indole, 2-(4-hydroxyphenyl)ethanol and tetrahexylammonium chloride. Particularly preferably, the aqueous system comprises MOPS or a mixture of MOPS with sodium salicylate and/or glucosamine hydrochloride.

The concentration of the substance used for the stabilization is dependent on the substance used. The concentration of 3-(N-morpholino)propanesulfonic acid in the aqueous system according to the invention is preferably from 1 to 1000 mmol/l, particularly preferably from 25 to 500 mmol/l, more preferably from 100 to 300 mmol/l and more particularly 200 mmol/l.

If the buffer contains sodium salicylate, the concentration of the sodium salicylate in the buffer is preferably 1-1000 mg/ml, particularly preferably from 25 to 500 mg/ml, more preferably from 200 to 300 mg/ml and more particularly 250 mg/ml. If the buffer contains hexaammonium heptamolybdate, the concentration of the hexaammonium heptamolybdate in the buffer is preferably from 1 to 300 mg/ml, particularly preferably from 50 to 250 mg/ml, more preferably from 100 to 200 mg/ml and more particularly 150 mg/ml. If the buffer contains glucosamine hydrochloride, the concentration of the glucosamine hydrochloride is preferably from 1 to 300 mg/ml, particularly preferably from 25 to 200 mg/ml, more preferably from 50 to 150 mg/ml and more particularly 100 mg/ml. If the buffer contains indole, the concentration of the indole is preferably from 1 to 300 mg/ml, particularly preferably from 25 bis 200 mg/ml, more preferably from 50 to 150 mg/ml and more particularly 100 mg/ml. If the buffer contains 2-(4-hydroxyphenyl)ethanol, the concentration of the indole is preferably from 1 to 300 mg/ml, particularly preferably from 25 to 200 mg/ml, more preferably from 50 to 150 mg/ml and more particularly 100 mg/ml. If the buffer contains tetrahexylammonium chloride, the concentration of the indole is preferably from 0.1 to 50 mg/ml, particularly preferably from 0.5 to 25 mg/ml, more preferably from 1 to 10 mg/ml and more particularly 5 mg/ml. If the buffer contains 1,2-dimethoxyethane, the proportion of the 1,2-dimethoxyethane in the buffer is preferably from 1 to 20 vol % (volume percent), preferably from 5 to 15 vol % and more particularly 10 vol %, corresponding to a concentration of the 1,2-dimethoxyethane of preferably from 8.7 to 174 mg/ml, particularly preferably from 43.5 to 130.5 mg/ml and more particularly 87 mg/ml. The substances mentioned in this paragraph are present in the aqueous system preferably in a concentration range of from 1 to 3000 mg/ml, more preferably from 25 to 2000 mg/ml, particularly preferably from 50 to 1500 mg/ml and more particularly from 400 to 850 mg/ml, based on the total concentration.

Furthermore, the solution/buffer according to the invention can comprise one or more further substances, preferably selected from the group containing pH regulators such as acids or bases, anticoagulants and organic solvents. For example, the buffer can contain weakly basic salts such as sodium acetate, preferably in a concentration of from 10 to 200 mmol/l, particularly preferably from 25 to 75 mmol/l. Useful anticoagulants are known to a person skilled in the art and comprise, for example, chelating agents such as heparin, citric acid, ethylendiamine-N,N,N′,N′-tetraacetic acid (EDTA) and 1,2-bis(2-aminophenoxy)ethane-N,N,N′,N′-tetraacetic acid (BAPTA) in the form of the acid, of an alkali metal salt or ester, which are capable of complexing divalent metal ions such as calcium ions. The concentration of such chelating agents in the buffer according to the invention is preferably from 2 to 100 mmol/l, particularly preferably from 2 to 50 mmol/l and more particularly from 5 to 15 mmol/l. In addition, the buffer according to the invention can contain organic solvents such as DMSO for example, preferably in a concentration of from 10 to 250 mmol/l, particularly preferably from 50 to 200 mmol/l.

The pH of the aqueous system according to the invention is preferably from 3 to 7, more preferably from 3.5 to 6.5, particularly preferably from 4 to 6 and more particularly from 4.5 to 5.5.

For the purposes of the invention, any sample which contains cells is referred to as a cell material-containing biological sample. The samples can, for example, be obtained from animal or plant tissues, tissue or cell cultures, bone marrow, human and animal body fluids such as blood, serum, plasma, urine, semen, cerebrospinal fluid, sputum and smears, plants, plant parts and plant extracts, for example juices, fungi, prokaryotic or eukaryotic microorganisms such as bacteria or yeasts, fossil or mummified samples, soil samples, sludge, wastewaters and foodstuffs. Preferably, the biological sample comprises blood, particularly preferably whole blood.

For the purposes of the invention, the term nucleic acids indicates both ribonucleic acids (RNA) and deoxyribonucleic acids (DNA). For the purposes of the invention, the abbreviations RNA and DNA indicate both an individual nucleic acid molecule (one nucleic acid) and a multiplicity of nucleic acids. Preferred nucleic acids for the purposes of the invention are all ribonucleic acids, more particularly messenger RNA (mRNA), transfer RNA (tRNA), ribosomal RNA (rRNA), heterogeneous nuclear RNA (hnRNA), so-called small nuclear RNA (snRNA), so-called small-interfering RNA (siRNA), microRNA (miRNA) and so-called antisense RNA.

The aqueous system according to the invention is capable of stabilizing nucleic acids, more particularly the unstable RNA in a sample, for at least 24 h at room temperature, preferably for at least three days at room temperature. For the purposes of the invention, the term room temperature preferably encompasses temperatures of 22±3° C. The storage life of the stabilized sample at temperatures below 20° C., for example at from 2 to 8° C., is correspondingly higher.

For the purposes of the invention, a sample is referred to as stabilized when the integrity of the contained nucleic acids after storage at a given temperature for a specified time is greater than the integrity of the nucleic acids in a (unstabilized) comparative sample which originates from the same source and was collected and stored under identical conditions, but without addition of a stabilization buffer. Preferably, the integrity of the nucleic acids in a sample which is referred to as stabilized for the purposes of the invention is, after storage for three days (t=72 h) at room temperature, at least 60% of the integrity of the nucleic acids at the time of admixing with the buffer according to the invention (t=0 h), particularly preferably at least 80% and more particularly at least 95%. Methods for determining the integrity of nucleic acids are known to a person skilled in the art. In the case of RNA, the aforementioned percentages are based on the RNA integrity number (RIN), the determination of which will be elaborated later in detail.

In addition, the buffers/solutions according to the invention are capable of minimizing the ex vivo gene expression in the stabilized samples compared to unstabilized samples and of thus preserving the in vivo transcription profile. Thus, the state of the cells at the time of sample collection can be largely maintained and be studied at a later time despite storage. Since cells can distinctly change especially the expression pattern (and thus transcription) outside their natural environment, this stabilization of the nucleic acids and the suppression of the ex vivo gene expression offers the possibility of storage of collected samples.

The invention further provides a method for stabilizing nucleic acids in cell material-containing biological samples while preserving the cell morphology of the cell material in order to provide samples for at least one of the following methods for analyzing the nucleic acids contained in the sample, without being restricted thereto: PCR, RT-PCR, electrophoretic methods, microarray analyses, labeling, isolation and/or detection of the nucleic acids, comprising the admixing of the biological sample with a nucleic acid-stabilizing aqueous system according to the invention.

The quicker the admixing of the sample, after collection from its natural environment, with the aqueous system according to the invention, the lesser the extent of the degradation of the nucleic acids contained in the sample and of the ex vivo gene induction or repression. Preferably, the sample is immediately admixed after collection from its natural environment with the buffer according to the invention. In a preferred embodiment, the sample is immediately transferred after collection from its natural environment to a vessel containing the aqueous system according to the invention. The volume used of the buffer/solution is in this case dependent on the sample. For the stabilization of whole blood samples, the sample is admixed with a volume of the buffer/solution which is preferably 1.5 to 10 times, particularly preferably 2 to 5 times, the volume of the whole blood sample.

The nucleic acids stabilized in the aqueous system according to the invention can be labeled, processed, isolated and detected according to known methods following storage. For this purpose, the cell material contained in the sample is first lysed, and the nucleic acids released in this process are isolated and, if necessary, purified by means of an appropriate method. The methods appropriate for this purpose are known to a person skilled in the art. The cell material can be lysed in, for example, the commercially available QIAzol Lysis Reagent from Qiagen (Hilden, Germany) in accordance with the QIAzol Handbook 10/2006. The RNA contained in the lysed sample can, for example, be removed from the DNA and the proteins by a phenol/chloroform extraction and be precipitated from the aqueous phase by subsequent precipitation with isopropanol. The RNA can be purified by means of, for example, the commercially available RNeasy Kits from Qiagen, or else with the aid of any other appropriate purification method.

The nucleic acids obtained can be subsequently further processed, for example reverse transcribed and amplified by means of RT-PCR methods in the case of RNA, amplified by means of PCR methods in the case of DNA and/or analyzed by means of electrophoretic methods such as Northern blotting (RNA) or Southern blotting (DNA) or so-called microarrays (Genechip analyses). For the purposes of the invention, the term RT-PCR encompasses in particular so-called quantitative RT-PCR methods (qRT-PCR or real-time RT-PCR), which allow quantification of the mRNA obtained.

The integrity of the RNA obtained was determined with the aid of electrophoretic methods. Firstly, classic gel electrophoreses were performed on denaturing agarose gels. In the case of intact RNA, such a gel shows, following staining with fluorescent dyes such as ethidium bromide or SYBR Green, two intensively fluorescing, sharply separated bands corresponding to the ribosomal RNAs 28 S and 18 S, and possibly further bands of lower intensity. The ratio of the fluorescent intensity of the 28 S rRNA band to the 18 S rRNA band is about 2:1 for intact RNA.

In addition, electrophoretic analyses of the RNA obtained were performed on microchips using the Agilent 2100 Bioanalyzer from Agilent. An algorithm implemented into the Bioanalyzer software was used to determine the RNA integrity number (RIN), which represents a system for quantifying RNA quality that considers not only the intensity of the 28 S and the 18 S rRNA band but also a range of further factors and thus allows a more reliable assessment of the integrity of the RNA than is possible with a purely visual estimation of the intensity on a gel. On the basis of the ribosomal subunits 28 S to 18 S rRNA and the ratio thereof, with degraded degradation products taken into account, a RIN value on a scale of from 1 to 10 is determined by the software. A numerical value of 1 corresponds here to completely degraded RNA, whereas a numerical value of 10 corresponds to completely intact RNA. The thus determined integrity of the rRNA is indicative of the integrity of the mRNA.

Furthermore, the buffers according to the invention do not lead to any qPCR inhibitors being introduced into the sample, or retained therein, during the processing. In addition, the buffers according to the invention are suitable for minimizing the ex vivo gene induction in the stabilized samples. This was demonstrated by means of qRT-PCR analyses of the RNA which was isolated according to known methods after storage of the sample in the buffers according to the invention. The results were quantified using the ΔΔC_{T} method, which is known to a person skilled in the art and in which the expression of the target genes (in the present case, c-fos and IL-1β) is normalized with that of a nonregulated reference gene (in the present case, 18 S rRNA was used).

Furthermore, the method according to the invention can comprise a step for immunohistologically labeling individual cell types in a sample containing various cell types, which labeling is carried out prior to analysis of the nucleic acids contained in the sample using the above-mentioned techniques, such as PCR, RT-PCR, electrophoretic methods or microarray analyses. Since the stabilization buffers of the present invention preserve the cell morphology of the cell material, the present invention allows the immunohistological labeling, analysis and/or separation of individual cell types before the nucleic acids contained in the cells are released by subsequent lysis of the cell material. The cells can, for example, be labeled and detected with specific antibodies even after two or more days of storage at room temperature. Since individual desired cell types can be specifically removed in this way from the multiplicity of further cell types contained in a biological sample, the subsequent analysis of the nucleic acids of the desired cell type, for example by techniques such as PCR, RT-PCR, electrophoretic methods or microarray analyses, is considerably simplified.

Preferably, the immunohistological labeling is carried out using fluorescently labeled antibodies, which allow UV/Vis spectroscopic detection of the antigen-antibody conjugate.

In a preferred embodiment, the method additionally contains a step for selecting and separating individual cell types from a sample containing various cell types, which step is carried out prior to the analysis of the nucleic acids contained in the sample with the aid of the above-mentioned methods, such as PCR, RT-PCR, electrophoretic methods or microarray analyses.

The method allows, for example, the flow-cytometric analysis of the stabilized cell material. Flow cytometry makes it possible to characterize a multiplicity of cells at the single-cell level with respect to their biochemical and physical properties within a very short time. For this reason, this technology is used routinely in, inter alia, hematology and immunology, for example for diagnosing and assessing the disease progression or therapeutic outcome of various diseases and viral infections, such as leukemia or HIV infections for example.

The principle of flow cytometry is based on the analysis of the optical properties of the cells which individually pass a laser beam in a measurement unit. Firstly, photomultipliers are used to analyze the light scattering and light refraction which are caused by a cell crossing the laser beam. The amount of scattered light is dependent on the size of the cell and the complexity thereof. For example, granulocytes, which have a rough surface, scatter distinctly more light than T lymphocytes, which have a smooth surface. The forward scatter (FSC) correlates with the volume of the cell, whereas the sideward scatter (SSC), measured at an angle of 90°, depends on the granularity of the cell, on the structure and size of the nucleus thereof, and on the amount of vesicles in the cell. Using these parameters, the cell types of blood can be differentiated into granulocytes, lymphocytes and monocytes. In addition, flow cytometry also allows the determination of the cell count in a sample and separation of the individual cell types.

Besides scattering, however, it is also possible to detect and quantify the emission of fluorescent dyes in a flow cytometer. In the case of fluorescence-based flow cytometry, often also called fluorescence-activated cell sorting (FACS), the cells are first labeled, prior to the flow-cytometric analysis, with a fluorescent dye which specifically binds to particular constituents of the cell. The intercalating dyes 4′,6-diamidino-2-phenylindole (DAPI) and propidium bromide bind, for example, to the DNA of a cell and enable the DNA content of the cell to be determined via the measurement of the fluorescence intensity. The cells can also be labeled with a specific antibody which either itself carries a fluorescent dye (direct immunofluorescence) or which is labeled with a fluorescently labeled secondary antibody in a second step after binding to the antigen (indirect immunofluorescence). By using CD3 antibodies labeled with the fluorescent dye fluorescein isothiocyanate (FITC) (CD3-FITC antibodies), it is possible, for example, to specifically label mature T lymphocytes and detect, count and separate them by flow cytometry. In addition, by using multiple laser sources, it is possible, when using multiple antibodies, to simultaneously detect various features and to use them as selection criteria for the subsequent separation of the labeled cells. Therefore, in the method according to the invention, the cell types are preferably selected and separated by means of fluorescence-based flow cytometry.

### DESCRIPTION OF THE FIGURES

FIG. 1 shows the electropherograms of two RNA samples which were incubated, in each case, for 24 hours, 3 days and 7 days at room temperature a) in an aqueous system according to the invention in the presence of lysed blood (left-hand column) and b) in an unstabilized aqueous solution without lysed blood (positive control, right-hand column). The RNA integrity number (RIN), which was determined with the aid of a software-implemented algorithm, is also displayed in the respective electropherogram.

FIG. 2 shows a comparison of the integrity of RNA which was isolated from blood samples which were stored for 2, 24 or 72 h in a MOPS buffer of pH 5 or in commercially available PAXgene or EDTA-containing sample vessels.

FIG. 3 shows the qRT-PCR-determined relative c-fos transcription profile of a sample which was stored in an EDTA-containing buffer (upper graph) compared to that of a sample which was stored in a MOPS buffer of pH 5 (lower graph).

FIG. 4 shows the qRT-PCR-determined relative IL-1β transcription profile of a sample which was stored in an EDTA-containing buffer (upper graph) compared to that of a sample which was stored in a MOPS buffer of pH 5 (lower graph).

FIG. 5 shows the electrophoresis gels of RNA which were obtained from whole blood samples after storage for 24 hours at room temperature. Shown on the far left is a figure of the electrophoresis gel of RNA which was isolated from an unstabilized stored blood sample, whereas the RNA analyzed in gels II to V was stored in stabilization buffers according to the invention (II: buffer containing MOPS, pH 5; III: buffer containing MOPS and 250 mg/ml sodium salicylate, pH 5; IV: buffer containing MOPS and 100 mg/ml glucosamine hydrochloride, pH 5; V: buffer containing MOPS and 250 mg/ml sodium salicylate and 100 mg/ml glucosamine hydrochloride, pH 5).

FIG. 6 shows the scattered light dot plots, obtained by means of flow-cytometric analyses, a) of a whole blood sample which was not stabilized according to the invention and which was stored beforehand for 24 h at 4° C. in a commercially available citrate buffer and b) of a whole blood sample which was stored beforehand for 24 h in a buffer according to the invention of pH 5 containing MOPS and 100 mg/ml glucosamine hydrochloride. In each case, shown on the left are the dot plots of the samples prior to staining with a specific antibody, shown in the middle are the dot plots after labeling of the leukocytes with CD3-FITC, and shown on the right are the histograms of the CD3-FITC-labeled leukocytes.

X-axis: forward scatter (FSC)
Y-axis: sideward scatter (SSC)

### EXAMPLES

### Example 1

### Stabilization of RNA in the Presence of Lysed Blood

The basis buffer used was a solution of 200 mmol/l MOPS, 50 mmol/l sodium acetate and 10 mmol/l EDTA in RNase-free water (pH 5). This basis buffer was admixed with the substances indicated in table 1. 800 μl of the different buffer compositions were admixed with 6 μg of RNA which had been isolated beforehand from Jurkat cells with the aid of the commercially available RNeasy Kit from Qiagen (Hilden, Germany), and incubated for a predefined time after addition of lysed blood containing free, activated RNases. As comparative sample (positive control), 6 μg of RNA were incubated without lysed blood in an unstabilized aqueous solution for the same period.

After, in each case, 1 hour, 1 day, and 3, 5 and 7 days, the samples were lysed using the QIAzol Lysis Reagent from Qiagen (Hilden, Germany) in accordance with the QIAzol Handbook 10/2006, and the RNA contained was isolated using an RNeasy Kit from Qiagen (Hilden, Germany). To this end, the sample was admixed with 2.5 ml of QIAzol Reagent from Qiagen (Hilden, Germany) and 500 μl of chloroform and centrifuged for 15 min at 12 000 rpm. The upper phase was carefully removed and admixed with 2.5 ml of ethanol. To bind the RNA to the RNeasy Mini column from Qiagen (Hilden, Germany), the column was loaded with the lysate and centrifuged for 1 min at 8000 rpm. 500 μl of Buffer RW1 from Qiagen (Hilden, Germany) were pipetted onto the column, and the column was centrifuged for 1 min at 8000 rpm. 80 μl of a mixture of 10 μl of DNase I solution and 70 μl of Buffer RDD from Qiagen (Hilden, Germany) were pipetted onto the column, and the column was incubated for 20 min at room temperature. Subsequently, 500 μl of Buffer RW1 from Qiagen (Hilden, Germany) were again pipetted onto the column, and the column was centrifuged for 1 min at 8000 rpm. The column was washed twice with 1 ml of Buffer RPE (for 1 min at 8000 rpm) and subsequently centrifuged for drying for 10 min at 14 000 rpm. To elute the RNA, 100 μl of RNase-free water were pipetted onto the column, the column was incubated for 1 min at room temperature and subsequently centrifuged for 1 min at 14 000 rpm. The eluate contained the purified RNA. This was detected over a denaturing agarose/formaldehyde gel by means of staining with SYBR Green. The buffers considered to be suitable for stabilization were those in which both 28 S rRNA and 18 S rRNA were still detectable on the agarose gel even after 3 days, preferably 5 days, particularly preferably 7 days, of storage in the presence of lysed blood. Specifically the buffers for which an intensity ratio of the two bands (28 S:18 S) of approximately 2:1 was maintained were referred to as stabilizing. In this connection, especially the solutions listed in table 1 were found to be suitable for stabilizing RNA in the presence of free, active RNases for longer than 24 h at room temperature.

TABLE 1

| Stabilization | |
|---|---|
| buffer | Composition |
| Buffer 1 | Basis buffer + 10 vol % 1,2-dimethoxyethane, pH 4 |
| Buffer 2 | 250 mg sodium salicylate per ml basis buffer |
| Buffer 3 | 150 mg hexaammonium heptamolybdate per ml basis buffer |
| Buffer 4 | 100 mg glucosamine hydrochloride per ml basis buffer |
| Buffer 5 | 100 mg indole per ml basis buffer |
| Buffer 6 | 100 mg 2-(4-hydroxyphenyl)ethanol per ml basis buffer |
| Buffer 7 | 5 mg tetrahexylammonium chloride per ml basis buffer |
| Buffer 8 | 250 mg sodium salicylate and 100 mg glucosamine |
| | hydrochloride per ml basis buffer |

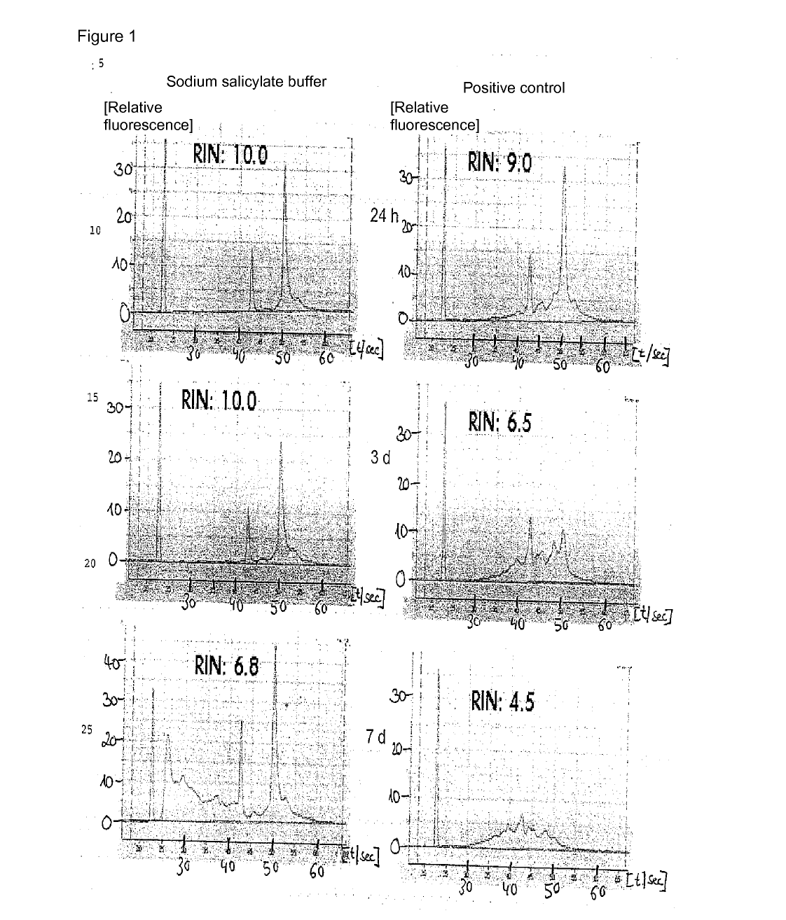

The integrity of the RNA obtained was determined with the aid of the Bioanalyzer 2100 from Agilent Technologies (Böblingen, Germany). As an example, FIG. 1 shows a comparison of the sample stabilized in buffer 2 with the unstabilized sample (positive control). In the electropherogram of the positive control, distinct noise in the baseline between the 18 S and the 28 S rRNA band at 43 s and 50 s respectively (the so-called inter-region), which is characteristic of RNA degradation in the sample, can already be seen after 24 h. The RIN of the positive control was only 9.0 after 24 h, whereas the RNA of the sample stabilized in the buffer 2 according to the invention had a RIN of 10.0.

In the positive control, after storage of the samples for three days at room temperature, the intensity of the 28 S band was already lower than the intensity of the 18 S band, and the RIN was only 6.5. In the sample stabilized with the buffer according to the invention, only a slight increase in the noise was to be seen, and the RIN was still 10.0. Even after storage for seven days at room temperature, the 18 S and the 28 S rRNA band are still identifiable in the stabilized sample as separate bands with an intensity ratio of 28 S:18 S=1.8 (RIN 6.8), whereas in the electropherogram of the control, a discrete 28 S band could no longer be seen (RIN 4.5). The values of the other buffer solutions listed in table 1 revealed similar good stabilization of the samples.

### Example 2

### Amplification of the Stabilized RNA by Means of Quantitative RT-PCR

To establish that the stabilization buffers according to the invention did not lead to any qPCR inhibitors being introduced into the sample, or retained therein, during the processing or the substances used in the buffers themselves acted as inhibitors, and that the buffers according to the invention are additionally suitable for minimizing the ex vivo gene induction, part of the RNA obtained was amplified using commercially available primers for GAPDH from Operon Biotechnology Inc. (Huntsville, Ala., USA) by means of a qRT-PCR in accordance with a standard protocol from Applied Biosystems Inc. (Foster City, Calif., USA).

To this end, part of the RNA (6 μg) obtained in example 1 was mixed with, in each case, 2.5 ml of lysed blood from three different donors and subsequently admixed with 5 ml of the basis buffer or added to commercially available PAXgene sample vessels and EDTA-containing sample vessels. Immediately after the mixing of the samples with the buffer (0 h), and after storage of the samples in the buffer for two hours (2 h), one day or three days (24 h or 72 h) at room temperature, the RNA contained in the samples was isolated using the QIAzol Lysis Reagent and the RNeasy Kit from Qiagen (Hilden, Germany) in accordance with the QIAzol Handbook 10/2006. All analyses were carried out in duplicate.

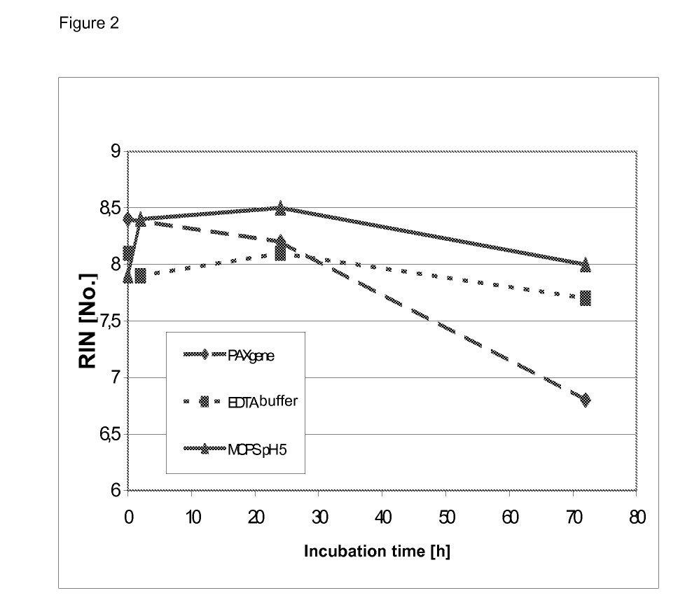

The integrity of the RNA obtained from a donor (donor 3) was determined with the aid of an Agilent Bioanalyzer 2100, as described in example 1. The results are shown in FIG. 2. This showed that the integrity of the RNA which was obtained from the sample stored in the buffer according to the invention is greater than the integrity of the RNA which was isolated from the samples stored in PAXgene or in EDTA.

The amount of the RNA isolated from 2.5 ml of blood was determined for the samples stored in the MOPS buffer or in EDTA, following dilution with water (factor of 7.5), by photometric determination of the light absorption at a wavelength of 260 nm. The purity of the RNA obtained is determined via the photometric determination of the ratio of the light absorption at 260 nm to that at 280 nm. The results are reported in table 2, and in each case, the mean values of the duplicates are reported.

TABLE 2

| | | | | Total yield |
|---|---|---|---|---|
| Donor | Method | Time [h] | A260/A280 | per 2.5 ml blood [mg] |
| 1 | EDTA | 0 | 2.1 | 4.36 |
| | | 2 | 2.05 | 8.55 |
| | | 24 | 2.2 | 9.44 |
| | | 72 | 2.1 | 5.97 |
| | MOPS pH 5 | 0 | 2.1 | 11.02 |
| | | 2 | 2.1 | 7.16 |
| | | 24 | 2.15 | 7.06 |
| | | 72 | 2.05 | 5.12 |
| 2 | EDTA | 0 | 1.9 | 5.45 |
| | | 2 | 1.95 | 4.26 |
| | | 24 | 2.05 | 3.40 |
| | | 72 | 1.9 | 2.94 |
| | MOPS pH 5 | 0 | 1.95 | 4.32 |
| | | 2 | 1.9 | 3.53 |
| | | 24 | 1.85 | 2.31 |
| | | 72 | 2.0 | 2.94 |
| 3 | EDTA | 0 | 2.0 | 12.05 |
| | | 2 | 2.05 | 10.16 |
| | | 24 | 2.1 | 7.99 |
| | | 72 | 2.0 | 6.17 |
| | MOPS pH 5 | 0 | 2.0 | 9.77 |
| | | 2 | 1.85 | 3.47 |
| | | 24 | 2.1 | 11.22 |
| | | 72 | 2.0 | 6.63 |

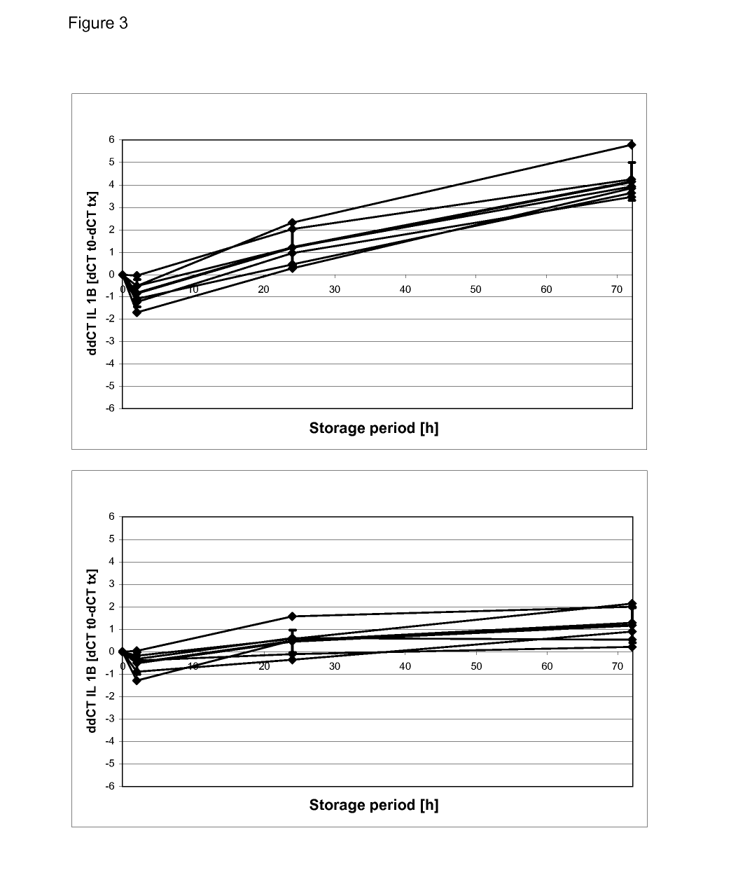

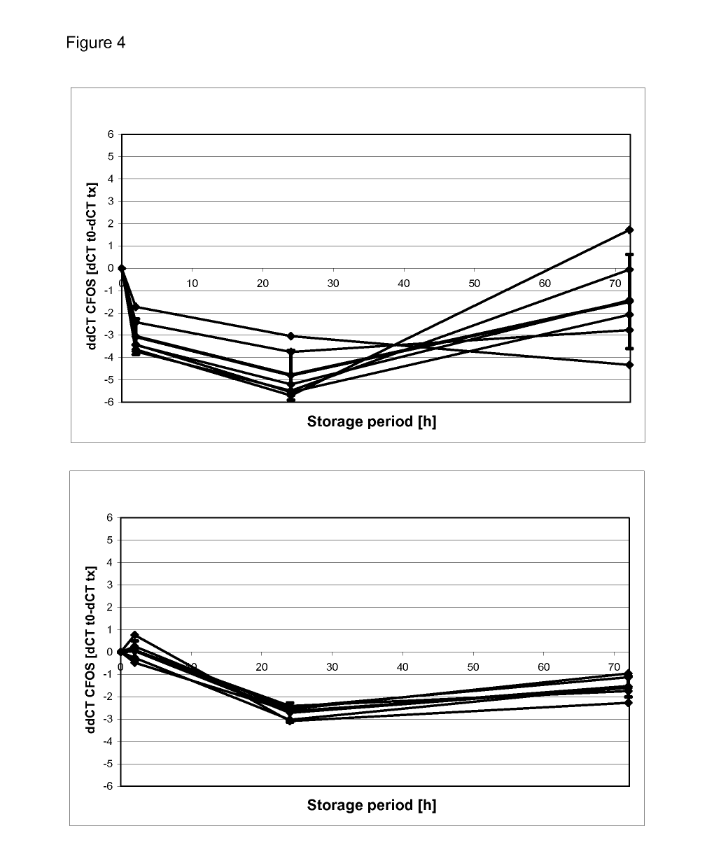

The relative expression of c-fos and IL-1β was analyzed relative to the expression of the 18 S rRNA for the samples reported in table 2 by means of the ΔΔC_{T} method. The results are summarized in tables 3 and 4 and clarified in FIGS. 3 and 4 and FIGS. 5 and 6, respectively.

TABLE 3
Effect of storage conditions on the transcription of c-fos

| | | Time | C_{T} | C_{T} (18 S | ΔC_{T} (18S- | ΔΔC_{T} |
|---|---|---|---|---|---|---|
| Donor | Method | [h] | (c-fos) | rRNA) | c-fos) | (t₀-tₓ) |
| 1 | EDTA | 0 | 27.27 | 25.43 | −1.84 | 0 |
| | | | 27.27 | 25.34 | −1.93 | 0 |
| | | 2 | 24.16 | 25.76 | 1.60 | −3.44 |
| | | | 24.04 | 25.53 | 1.49 | −3.42 |
| | | 24 | 22.47 | 25.83 | 3.36 | −5.20 |
| | | | 22.29 | 25.91 | 3.62 | −5.55 |
| | | 72 | 27.89 | 27.48 | −0.41 | −1.43 |
| | | | 25.71 | 25.86 | 0.15 | −2.08 |
| | MOPS pH 5 | 0 | 26.89 | 25.88 | −1.01 | 0 |
| | | | 27.06 | 25.18 | −1.88 | 0 |
| | | 2 | 26.63 | 25.37 | −1.26 | 0.25 |
| | | | 27.01 | 25.61 | −1.40 | −0.48 |
| | | 24 | 24.21 | 25.76 | 1.55 | −2.56 |
| | | | 24.70 | 25.46 | 0.76 | −2.64 |
| | | 72 | 27.35 | 27.30 | −0.05 | −0.96 |
| | | | 26.00 | 25.73 | −0.27 | −1.61 |
| 2 | EDTA | 0 | 26.76 | 25.43 | −1.33 | 0 |
| | | | 27.72 | 25.02 | −2.70 | 0 |
| | | 2 | 24.78 | 25.18 | 0.40 | −1.73 |
| | | | 24.92 | 24.64 | −0.28 | −2.42 |
| | | 24 | 23.03 | 24.74 | 1.71 | −3.04 |
| | | | 23.12 | 24.17 | 1.05 | −3.75 |
| | | 72 | 24.56 | 27.56 | 3.00 | −4.33 |
| | | | 24.68 | 24.75 | 0.07 | −2.77 |
| | MOPS pH 5 | 0 | 27.17 | 24.66 | −2.51 | 0 |
| | | | 26.70 | 24.22 | −2.48 | 0 |
| | | 2 | 27.19 | 24.94 | −2.25 | −0.26 |
| | | | 27.76 | 24.52 | −3.24 | 0.76 |
| | | 24 | 24.14 | 24.66 | 0.52 | −3.03 |
| | | | 24.18 | 24.79 | 0.61 | −3.09 |
| | | 72 | 25.57 | 24.61 | −0.96 | −1.55 |
| | | | 25.51 | 25.30 | −0.21 | −2.27 |
| 3 | EDTA | 0 | 26.51 | 24.08 | −2.43 | 0 |
| | | | 27.45 | 25.02 | −2.43 | 0 |
| | | 2 | 23.93 | 25.14 | 1.21 | −3.64 |
| | | | 23.56 | 24.86 | 1.30 | −3.73 |
| | | 24 | 21.41 | 24.68 | 3.27 | −5.70 |
| | | | 21.97 | 25.03 | 3.06 | −5.49 |
| | | 72 | 28.53 | 24.38 | −4.15 | 1.72 |
| | | | 26.49 | 24.12 | −2.37 | −0.06 |
| | MOPS pH 5 | 0 | 27.20 | 25.52 | −1.68 | 0 |
| | | | 27.51 | 25.27 | −2.24 | 0 |
| | | 2 | 26.11 | 24.39 | −1.72 | 0.04 |
| | | | 27.34 | 25.02 | −2.32 | 0.08 |
| | | 24 | 23.98 | 24.77 | 0.79 | −2.47 |
| | | | 24.51 | 24.68 | 0.17 | −2.41 |
| | | 72 | 25.69 | 25.14 | −0.55 | −1.13 |
| | | | 25.71 | 25.22 | −0.49 | −1.75 |

TABLE 4
Effect of storage conditions on the transcription of IL-1β

| | | Time | C_{T} | C_{T} (18 S | ΔC_{T} (18S- | ΔΔC_{T} |
|---|---|---|---|---|---|---|
| Donor | Method | [h] | (IL-1β) | rRNA) | IL-1β) | (t₀-tₓ) |
| 1 | EDTA | 0 | 24.53 | 22.34 | −2.19 | 0 |
| | | | 24.68 | 22.30 | −2.38 | 0 |
| | | 2 | 24.17 | 23.08 | −1.09 | −1.10 |
| | | | 24.09 | 23.40 | −0.69 | −1.69 |
| | | 24 | 25.11 | 22.49 | −2.62 | 0.43 |
| | | | 25.21 | 22.56 | −2.65 | 0.27 |
| | | 72 | 29.13 | 23.31 | −5.82 | 3.63 |
| | | | 29.26 | 23.04 | −6.22 | 3.84 |
| | MOPS pH 5 | 0 | 24.87 | 22.18 | −2.69 | 0 |
| | | | 25.08 | 22.51 | −2.57 | 0 |
| | | 2 | 24.33 | 21.97 | −2.36 | −0.33 |
| | | | 24.31 | 22.14 | −2.17 | −0.40 |
| | | 24 | 25.48 | 22.18 | −3.30 | 0.61 |
| | | | 25.35 | 22.89 | −2.46 | −0.11 |
| | | 72 | 27.34 | 24.13 | −3.21 | 0.52 |
| | | | 26.22 | 23.44 | −2.78 | 0.21 |
| 2 | EDTA | 0 | 26.21 | 21.72 | −4.49 | 0 |
| | | | 26.29 | 22.08 | −4.21 | 0 |
| | | 2 | 26.35 | 22.37 | −3.98 | −0.51 |
| | | | 26.51 | 22.34 | −4.17 | −0.04 |
| | | 24 | 28.79 | 21.98 | −6.81 | 2.32 |
| | | | 28.43 | 22.19 | −6.24 | 2.03 |
| | | 72 | 32.53 | 22.24 | −10.29 | 5.60 |
| | | | 32.28 | 23.84 | −8.44 | 4.23 |
| | MOPS pH 5 | 0 | 25.51 | 21.54 | −3.97 | 0 |
| | | | 26.03 | 21.72 | −4.31 | 0 |
| | | 2 | 25.80 | 21.81 | −3.99 | 0.02 |
| | | | 25.72 | 21.58 | −4.14 | −0.17 |
| | | 24 | 27.22 | 21.67 | −5.55 | 1.58 |
| | | | 26.99 | 22.10 | −4.89 | 0.58 |
| | | 72 | 27.53 | 21.57 | −5.96 | 1.99 |
| | | | 28.02 | 21.55 | −6.47 | 2.16 |
| 3 | EDTA | 0 | 24.84 | 23.64 | −1.20 | 0 |
| | | | 25.45 | 23.43 | −2.02 | 0 |
| | | 2 | 24.47 | 23.78 | −0.69 | −0.51 |
| | | | 24.44 | 23.65 | −0.79 | −1.23 |
| | | 24 | 26.43 | 24.02 | −2.41 | 1.21 |
| | | | 26.61 | 23.65 | −2.96 | 0.94 |
| | | 72 | 29.37 | 24.23 | −5.14 | 3.94 |
| | | | 29.80 | 24.31 | −5.49 | 3.47 |
| | MOPS pH 5 | 0 | 25.22 | 23.62 | −1.60 | 0 |
| | | | 25.14 | 23.48 | −1.66 | 0 |
| | | 2 | 24.13 | 23.83 | −0.30 | −1.30 |
| | | | 24.53 | 23.78 | −0.75 | −0.91 |
| | | 24 | 25.46 | 23.35 | −2.11 | 0.51 |
| | | | 24.88 | 23.58 | −1.30 | −0.36 |
| | | 72 | 26.28 | 23.38 | −2.90 | 1.30 |
| | | | 26.12 | 23.56 | −2.56 | 0.90 |

The C_{T} values for c-fos and IL-1β and for 18 S rRNA at time t=0 h are comparable in each case for both buffers. This shows that the use of the MOPS buffer did not lead to any qPCR inhibitors being introduced into the sample, or retained therein, during the processing or the substances used in the buffers themselves acted as inhibitors.

The c-fos transcription level of the EDTA-stored samples initially fell strongly in the case of a storage time of up to 24 h (ΔΔC_{T} values of up to −5.55), implying degradation of the RNA, but then rose strongly with a longer storage period, presumably because of ex vivo gene induction. By contrast, in the MOPS-stabilized samples, the c-fos transcription level fell distinctly less strongly, and an appreciable ex vivo gene induction was also not observed within a period of 24 h.

In connection with the IL-1β transcription level, a distinct increase in the ΔΔC_{T} values over time was also observed in the EDTA-stored samples, whereas the transcription level in the MOPS-stabilized samples rose to a distinctly lesser extent even after 72 h.

### Example 3

### Microscopic Examination of the Cell Material in Whole Blood Samples after Stabilization

2.5 ml of blood were admixed with 5 ml of different stabilization buffers and incubated for 24 h at room temperature. The blood samples, wherein one a) was not admixed with a stabilization buffer, one b) was incubated in a MOPS buffer solution of pH 4, one c) was incubated in the basis buffer (MOPS, pH 5), one d) was incubated in a 1,2-dimethoxyethane-containing buffer (buffer 1 in table 1), one e) was incubated in a sodium salicylate-containing buffer (buffer 2), one f) was incubated in a glucosamine hydrochloride-containing buffer (buffer 4) and one g) was incubated in a sodium salicylate- and glucosamine hydrochloride-containing buffer (buffer 8), were subsequently centrifuged for 5 min at 1000×g, and the supernatant was decanted up to 2.5 ml. In order to be able to assess the quality of the cell material, the samples were subsequently examined under the microscope. When viewing the cells under the microscope, it was possible to see that the cells which were stored in the buffers according to the invention were still intact even after storage for 24 h at RT, whereas the nonstabilized cells no longer corresponded to the original cell morphology to a distinctly identifiable extent and were in some cases lysed. Especially in buffers b) and c), e) and g), it was even possible to see intact granulocytes.

### Example 4

### Lysis of the Cells and Gel Electrophoretic Analysis of the RNA

Further samples stored for 24 h in different stabilization buffers according to table 1, just like in example 3, were lysed using the QIAzol Lysis Reagent in accordance with the known protocols. The RNA contained in the lysed sample was isolated by means of a phenol/chloroform extraction and purified by means of an RNeasy Kit (Qiagen). Subsequently, the purified RNA was analyzed by means of a denaturing agarose gel following staining with SYBR Green. All analyses were carried out twice.

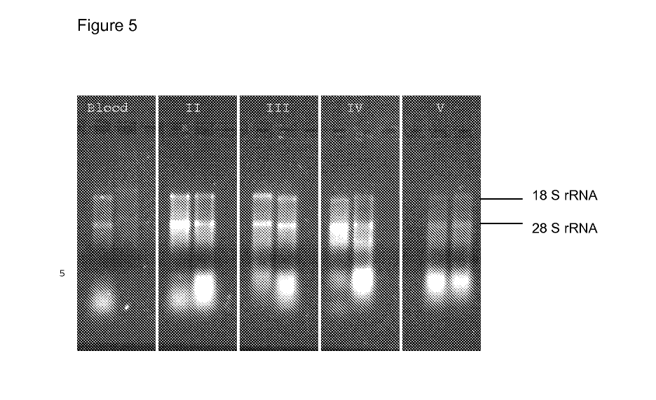

The results of the gel electrophoresis are shown in FIG. 5. Shown on the far left is a figure of the electrophoresis gel of the RNA which was isolated from a blood sample stored unstabilized for 24 h at room temperature, whereas the RNA analyzed in gels II to V was stored in stabilization buffers according to the invention (II: basis buffer; III: buffer 2, pH 5; IV: buffer 4, pH 5; V: buffer 8). Whereas the RNA obtained from the blood sample stored unstabilized is only still weakly identifiable in the gel and the intensity ratio of the 28 S rRNA band to the 18 S rRNA band is also already distinctly lower than 2:1, both bands are distinctly identifiable in a 28 S:18 S signal ratio of about 2:1 in the gels of the RNA obtained from the stabilized samples.

### Example 5

### Fluorescence-Activated Cell Sorting (FACS) of the Stabilized Cells

2.5 ml of blood were in each case admixed with 5 ml of the various stabilization buffers according to table 1 and incubated for 24 h at room temperature. The cells contained in the sample were subsequently labeled using a CD3-FITC antibody in accordance with a standard protocol (admixing of the sample with 10-20 vol % CD3-FITC, incubation of the mixture in the dark at room temperature, lysis of the erythrocytes, centrifugation, washing with PBS buffer, recentrifugation, admixing of the sample with 1% paraformaldehyde solution in PBS) and analyzed by flow cytometry with the aid of a FACSCalibur from BD Biosciences (San Jose, Calif., USA).

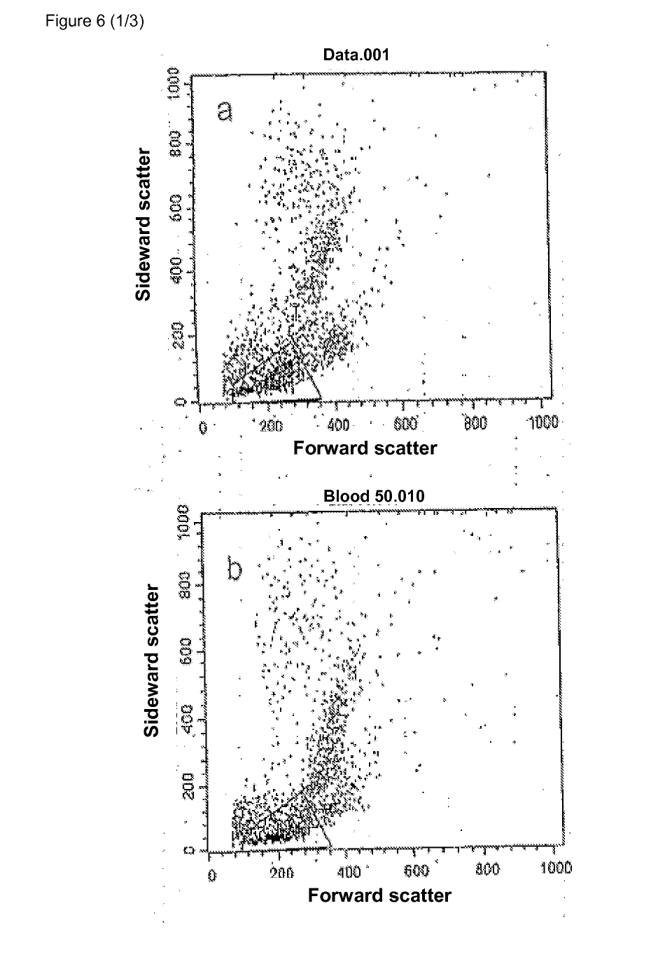

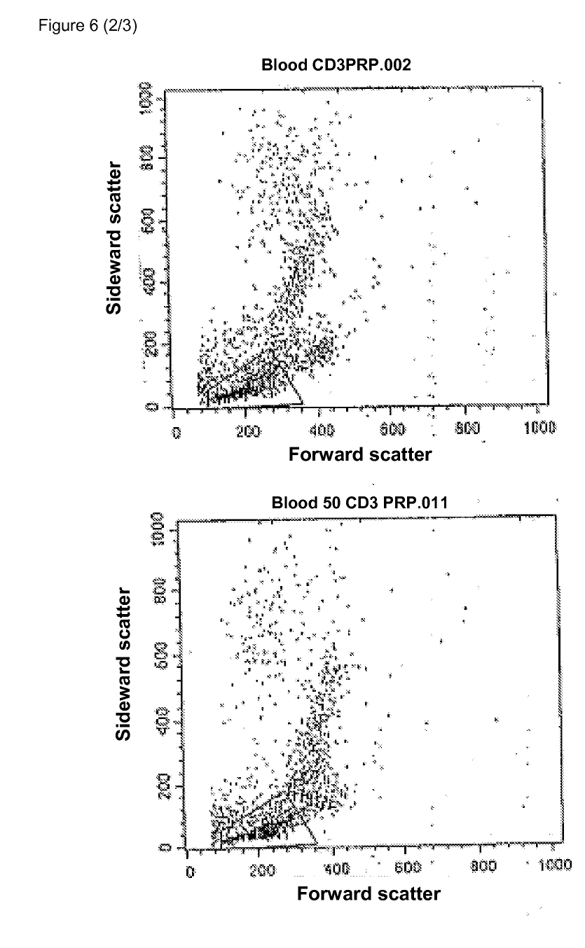

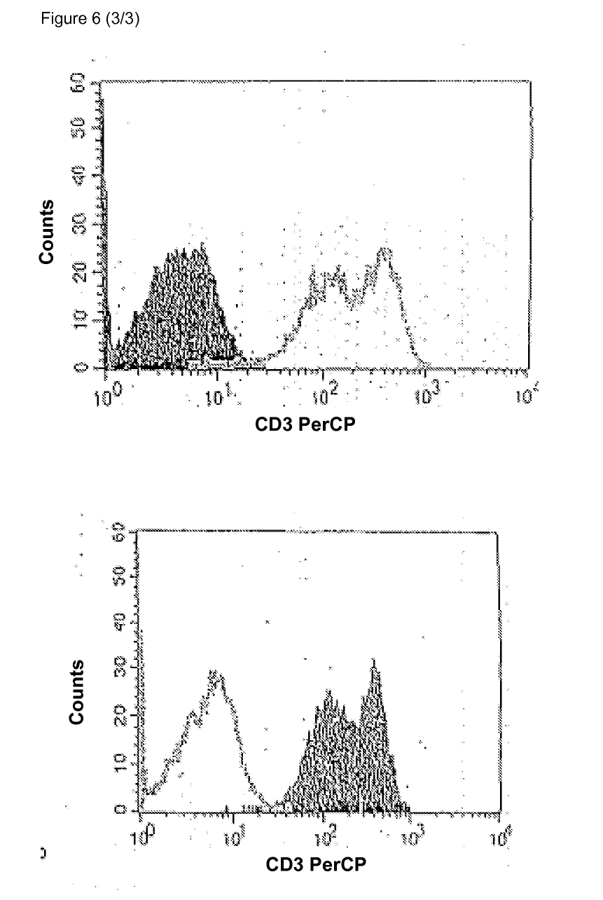

In this connection, especially buffer solutions of pH 5 containing (i) MOPS (basis buffer), (ii) MOPS and 250 mg/ml sodium salicylate (buffer 2), (iii) MOPS and 100 mg/ml glucosamine hydrochloride (buffer 4) and (iv) 10 vol % 1,2-dimethoxyethane in a MOPS-containing buffer (buffer 1) were found to be very well suited for stabilizing the whole blood samples at room temperature while preserving the cell morphology, and so, even after storage of the sample, cell-specific labeling of the cells with antibodies and subsequent fluorescence-activated cell sorting was possible. This is clarified in FIG. 6 by a comparison of a nonstabilized sample (a), which was stored in a citrate buffer for 24 h at 4° C., with a sample which was incubated in the glucosamine hydrochloride-containing MOPS buffer for 24 h (b). Shown on the left are the respective dot plots, obtained by FACS analysis, of the blood samples prior to labeling with CD3-FITC and shown in the middle are the respective dot plots of the CD3-FITC-labeled blood samples, which exhibit the pattern typical of leukocytes. It can be seen here that the system according to the invention does not lyse the cells. The right-hand figures show the histograms obtained for samples a) and b) following labeling of the leukocytes with CD3-FITC. It can be seen unambiguously that the cells stored in the buffer according to the invention are intact, and so they can be stained with specific antibodies and analyzed by FACS analysis.

X-axis: forward scatter (FSC)
Y-axis: sideward scatter (SSC)

1.-14. (canceled)

15. A method for stabilizing nucleic acids in a cell material-containing biological sample while preserving the cell morphology of the cell material, comprising:
admixing the biological sample with an aqueous system that comprises one or more substances selected from the group consisting of 3-(N-morpholino)propanesulfonic acid (MOPS), 1,2-dimethoxyethane, sodium salicylate, hexaammonium heptamolybdate, glucosamine hydrochloride, indole, 2-(4-hydroxyphenyl)ethanol, and tetrahexylammonium chloride.

16. The method of claim 15, wherein the aqueous system comprises MOPS or a mixture of MOPS and at least one further substance selected from the group consisting of 1,2-dimethoxyethane, sodium salicylate, hexaammonium heptamolybdate, glucosamine hydrochloride, indole, 2-(4-hydroxyphenyl)ethanol, and tetrahexylammonium chloride.

17. The method of claim 16, wherein the aqueous system comprises MOPS or a mixture of MOPS with sodium salicylate and/or glucosamine hydrochloride.

18. The method of claim 16, wherein the aqueous system comprises at least one further substance selected from the group consisting of anticoagulants and organic solvents.

19. The method of claim 15, wherein the substances present in the aqueous system are present in a concentration range of from 1 to 3000 mg/ml.

20. The method of claim 19, wherein the substances present in the aqueous system are present in a concentration range of from 25 to 2000 mg/ml.

21. The method of claim 20, wherein the substances present in the aqueous system are present in a concentration range of from 50 to 1500 mg/ml.

22. The method of claim 21, wherein the substances present in the aqueous system are present in a concentration range of from 400 to 850 mg/ml.

23. The method of claim 22, wherein the aqueous system is a buffer, and the pH of the buffer is from 3 to 7.

24. The method of claim 23, wherein the pH of the buffer is from 4 to 6.

25. The method of claim 24, wherein the pH of the buffer is from 4.5 to 5.5.

26. The method of claim 15, wherein the biological sample comprises blood.

27. The method of claim 26, wherein the biological sample comprises whole blood.

28. The method of claim 15, wherein the nucleic acids are ribonucleic acids (RNA).

29. The method of claim 15, further comprising analyzing the nucleic acids contained in the sample using at least one of the methods selected from the group consisting of PCR, RT-PCR, electrophoresis, microarray analyses, and labelling, isolation and/or detection of the nucleic acids.

30. The method of claim 29, wherein the sample contains multiple individual cell types, and wherein the method further comprises immunohistologically labelling the multiple individual cell types in the sample prior to analyzing the nucleic acids contained in the sample.

31. The method of claim 30, wherein the cell types are labelled with fluorescently labelled antibodies.

32. The method of claim 29, wherein the sample contains multiple individual cell types, and wherein the method further comprises selecting and separating the cell types prior to analyzing the nucleic acids contained in the sample.

33. The method of claim 32, wherein the cell types are selected and separated by means of fluorescence-based flow cytometry.

34. A kit for stabilizing, isolating or stabilizing and isolating nucleic acids from a cell material-containing sample, comprising an aqueous system that comprises one or more substances selected from the group consisting of 3-(N-morpholino)propanesulfonic acid (MOPS), 1,2-dimethoxyethane, sodium salicylate, hexaammonium heptamolybdate, glucosamine hydrochloride, indole, 2-(4-hydroxyphenyl)ethanol, and tetrahexylammonium chloride.
